(19) United States
(12) Patent Application Publication
(10) Pub. No.: US 2009/0111169 A1
(43) Pub. Date: Apr. 30, 2009

(54) BIOCHIP AND METHOD OF FABRICATION

(75) Inventors: Won-Sun KIM, Gyeonggi-do (KR); Jung-Hwan HAH, Gyeonggi-do (KR); Sung-Min CHI, Gyeonggi-do (KR); Sun-Ok JUNG, Gyeonggi-do (KR); Man-Hyoung RYOO, Gyeonggi-do (KR)

Correspondence Address: MARGER JOHNSON & MCCOLLOM, P.C., 210 SW MORRISON STREET, SUITE 400, PORTLAND, OR, 97204, US

(73) Assignee: Samsung Electronics Co., Ltd., Gyeonggi-do (KR)

(21) Appl. No.: 12/209,876

(22) Filed: Sep. 12, 2008

(30) Foreign Application Priority Data
Sep. 13, 2007 (KR) 10-2007-0093281

(51) Int. Cl. C12M 1/34

(52) U.S. Cl. 435/287.1

(57) ABSTRACT

A biochip and method of fabricating the same are provided. The biochip can include a substrate, a plurality of active pads formed on the substrate, each of the plurality of active pads having a surface roughness and being patterned so as to produce photonic crystals, and a plurality of probes directly or indirectly coupled with at least some of the plurality of active pads.

### CROSS-REFERENCE TO RELATED APPLICATION

This application claims priority to Korean Patent Application No. 10-2007-0093281, filed Sep. 13, 2007, the disclosure of which is incorporated herein by reference in its entirety.

### BACKGROUND

1. Technical Field

The disclosed technology relates to a biochip and method of fabricating the same, and more particularly, to a biochip for analyzing components of a bio sample using probes, and a method of fabricating the same.

2. Description of Related Art

Biochips exemplified by microarrays analyze specific components of biological samples by providing the biological samples to probes of a substrate immobilized on a substrate and observing reactions occurring between probes and the biological samples. A wide variety of different kinds of probes are immobilized on a biochip for each cell, so that a large amount of data can be read in one cycle of an experiment. Today, the rapid advance of high integration technology has allowed the collection of vast amounts of data.

As the quantity of data to be analyzed increases, high integration of biochips is required. However, in order to attain highly integrated biochips, it is necessary to reduce a design rule. Reducing a design rule means a reduction in the area occupied by a probe cell, that is, a reduction in the number of probes coupled to a probe cell. The reduced number of probes makes it difficult to ensure sufficient detection intensity. In addition, as the design rule is reduced, the resolution in data analysis may be considerably lowered due to fluorescence interference between different probes adjacent to each other.

### SUMMARY

The disclosed technology provides a biochip which can increase the number of probes coupled thereto for each probe cell.

The disclosed technology also provides a biochip which can increase the detection intensity and improve the resolution.

The disclosed technology provides a biochip substrate which can increase the number of probes coupled thereto for each probe cell.

The disclosed technology provides a biochip substrate which can increase the detection intensity and improve the resolution.

The disclosed technology provides a method of fabricating the biochip.

The above and other objects of the disclosed technology will be described in or be apparent from the following description of various embodiments.

Certain embodiments provide a biochip including a substrate, a plurality of active pads formed on the substrate, each of the plurality of active pads having a surface roughness and being patterned so as to produce photonic crystals, and a plurality of probes directly or indirectly coupled with at least some of the plurality of active pads.

Other embodiments provide a biochip including a substrate, a plurality of active pads or an active layer formed on the substrate, including a surface unevenness having a surface roughness of a Root Mean Square (RMS) within a range of about 0.2 to about 5 nm, and a plurality of probes directly or indirectly coupled with at least a portion of the plurality of active pads.

Still other embodiments provide a biochip substrate including a plurality of active pads having a surface unevenness, wherein the plurality of active pads are patterned so as to produce photonic crystals, and wherein the plurality of active pads are capable of being directly or indirectly coupled to functional groups that are coupled to probes.

Yet other embodiments provide a biochip substrate including a plurality of active pads or an active layer including a surface unevenness including a surface roughness having a Root Mean Square (RMS) of about 0.2 to about 5 nm, wherein the plurality of active pads is capable of producing functional groups directly or indirectly coupled to probes attached to a surface of the biochip substrate.

Further embodiments provide a method of fabricating a biochip including forming a plurality of active pads formed on a substrate, having a surface unevenness and patterned so as to produce photonic crystals, and directly or indirectly coupling a plurality of probes to some or all of the plurality of active pads.

Still further embodiments provide a method of fabricating a biochip including forming a plurality of active pads or an active layer formed on a substrate, including a surface unevenness having a surface roughness of an RMS ranging from about 0.2 to about 5 nm, and directly or indirectly coupling a plurality of probes to some or all of the plurality of active pads.

### BRIEF DESCRIPTION OF THE DRAWINGS

The above and other features and advantages of the disclosed technology will become more apparent by describing in detail various embodiments thereof with reference to the attached drawings in which:

FIG. 1 is a sectional view of a biochip according to a first embodiment of the disclosed technology;

FIG. 2 is a layout view of the biochip according the first embodiment of the disclosed technology;

FIG. 3 is a sectional view of a biochip according to a second embodiment of the disclosed technology;

FIG. 4 is a sectional view of a biochip according to a third embodiment of the disclosed technology;

FIG. 5 is a layout view of the biochip according the third embodiment of the disclosed technology;

FIG. 6 is a sectional view of a biochip according to a fourth embodiment of the disclosed technology;

FIGS. 7 through 11 are sectional views of intermediate structures illustrating a method of fabricating the biochip illustrated in FIG. 1, according to the first embodiment of the disclosed technology;

FIGS. 12 and 13 are sectional views of intermediate structures illustrating another method of fabricating the biochip illustrated in FIG. 1, according to the first embodiment of the disclosed technology; and

FIGS. 14 and 15 are sectional views of intermediate structures illustrating still another method of fabricating the biochip illustrated in FIG. 1, according to the first embodiment of the disclosed technology.

### DETAILED DESCRIPTION

Advantages and features of the disclosed technology and methods of accomplishing the same may be understood more readily by reference to the following detailed description of various embodiments and the accompanying drawings. The disclosed technology may, however, be embodied in many different forms and should not be construed as being limited to the embodiments set forth herein. Rather, these embodiments are provided so that this disclosure will be thorough and complete and will fully convey various concepts of the disclosed technology to those skilled in the art, and the present invention will only be defined by the appended claims. Accordingly, in order to avoid obscuring the invention, in some specific embodiments, well known processing steps, structures, and techniques have not been described in detail.

It is noted that the use of any and all examples, or exemplary terms provided herein, is intended merely to better illuminate the invention and is not a limitation on the scope of the invention unless otherwise specified. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein the term “and/or” includes any and all combinations of one or more of the associated listed items.

The disclosed technology will be described with reference to perspective views, cross-sectional views, and/or plan views, in which various embodiments of the disclosed technology are shown. Thus, the profile of an exemplary view may be modified according to manufacturing techniques and/or allowances. That is, the embodiments of the disclosed technology are not intended to limit the scope of the present invention but cover all changes and modifications that can be caused due to a change in manufacturing process. In the drawings, various components may be exaggerated or reduced for clarity. Like reference numerals refer to like elements throughout the specification.

Embodiments of the disclosed technology will now be described with reference to the accompanying drawings.

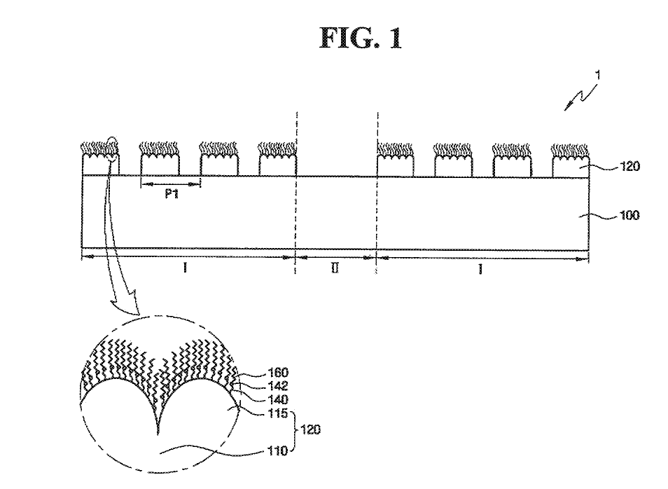

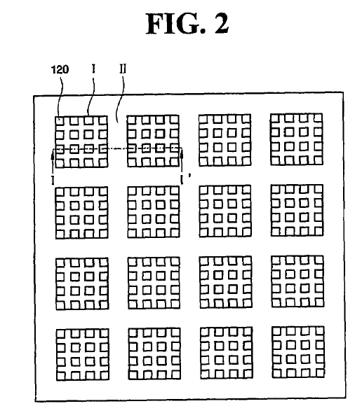

FIG. 1 is a sectional view of a biochip according to a first embodiment of the disclosed technology, and FIG. 2 is a layout view of an active pad of the biochip shown in FIG. 1.

Referring to FIGS. 1 and 2, the biochip 1 according to a first embodiment of the disclosed technology includes a substrate 100, a plurality of active pads 120 on the substrate 100, and a plurality of probes 160 coupled to the active pads 120.

Each of the plurality of active pads 120 includes a surface unevenness or roughness 115 to increase a surface area for coupling of the probes 160. In a case of employing a method of fabricating the surface unevenness 115 which will be described below, the Root Mean Square (RMS) of the surface roughness 115 may range from about 0.2 to about 5 nm, for example, but is not limited thereto. A fabricating method can be employed to form the surface roughness 115 of the active pads 120.

When the active pads 120 are patterned so as to produce photonic crystals, additional effects of increasing the detection intensity and improving the resolution can be achieved. The photonic crystals are two or more regularly arranged molecules having different refraction indexes or dielectric constants and are capable of producing photonic bandgaps to prevent electromagnetic waves having a particular frequency or wavelength from being propagated into the photonic crystals. The photonic crystal structure having photonic bandgaps can serve as an optical filter that reflects (or transmits) only the light in a particular wavelength range for amplification (or attenuation).

The principle of light propagation in the photonic crystal structure is similar to Bragg X-ray scattering by periodically patterned atoms.

Accordingly, the active pads 120 function as a first dielectric material and air filling a space between the active pads 120 functions as a second dielectric material. When a pitch P1 of the active pads 120 becomes n-fourth, (i.e., n/4) times of a fluorescence wavelength (λ) for detection, where n is an integer, the fluorescence wavelength is selectively reflected and amplified. For example, if the fluorescence wavelength is in a range of about 400 nm to about 500 nm, the pitch P1 of the active pads 120 may be in a range of about 200 nm to about 250 nm, for example, but is not limited thereto.

A thickness of each of the active pads 120 is also contributable to improvement of the resolution as long as it becomes n-fourth (i.e., n/4) times of a fluorescence wavelength (λ) for detection, where n is an integer. The thickness of each of the active pads 120 is, in one embodiment, in a range of about 100 nm to about 125 nm.

The activation pads 120 may be made of materials that can provide functional groups by being directly or indirectly coupled to the probes 160. Alternatively, the activation pads 120 may be made of materials that can provide the functional groups by a variety of surface treatments, such as annealing, ozonolysis, acid treatment, or base treatment. The phrase “being directly coupled to the probes 160” means being coupled to the probes 160 without other intermediate substance, and the phrase “being indirectly coupled” means that being coupled to the probes 160 by means of the linkers 140.

The surface unevenness 115 forming the active pads 120 may be formed of silicon dots or hemispherical silicon grains (HSGs). When the surface unevenness 115 is formed of silicon dots, a base 110 of the active pads 120 may be made of silicon oxide. When the surface unevenness 115 is formed of hemispherical silicon grains (HSGs), the base 110 of the active pads 120 may be polysilicon or amorphous silicon. The surface unevenness 115 may produce functional groups by annealing or acid treatment.

The biochip 1 includes a plurality of probe cell regions I to which probes are coupled, and non-probe cell regions II to which probes are not coupled. While the probes 160 of the same sequences are immobilized on a probe cell region I, the probes 160 of different sequences may be immobilized on different probe cell regions I.

The different probe cell regions I are separated from each other by the non-probe cell region II. Thus, each probe cell region I is surrounded by the non-probe cell regions II, and the non-probe cell regions II may be connected to one another into a single unit. The plurality of probe cell regions I may be arranged in a matrix configuration. In certain embodiments, the matrix configuration may have a regular pitch.

Referring to FIGS. 1 and 2, the active pads 120 may be formed only on the probe cell regions I. In this case, the probe cell regions I are physically and chemically separated by means of the non-probe cell regions II containing no functional group capable of being coupled to the probes 160. In some embodiments, the linkers are coupled only to the physically and chemically separated, three-dimensional active pads 120. In other embodiments, chemically separated characteristics of the probe cell regions I can be further added. As described above, if there are physically and chemically separated probe cell regions I, the probes 160 cannot be coupled to the non-probe cell regions II at all, thereby noticeably increasing a signal to noise ratio. Accordingly, analysis accuracy can be enhanced. While FIG. 2 illustrates, by way of example, that 16 active pads 120 are formed on a probe cell region I, a biochip may include more than 16 active pads 120 in practical applications. For example, assuming that a length of a side of each probe cell region I is 10 μm, and a length of a side of each of the active pads 120 is 200 nm, about 2500 active pads 120 may be arranged in a single probe cell region I.

The substrate 100 may be made of a material capable of minimizing or substantially preventing unwanted non-specific bonds during biological sample assay, e.g., hybridization. Further, the substrate 100 may be made of a material capable of transmitting visible and/or UV light. The substrate 100 may be a flexible substrate or a rigid substrate. The flexible substrate may be a membrane of nylon or cellulose and a plastic film. The rigid substrate may be a silicon substrate, a quartz substrate, a glass substrate made of soda-lime glass, or a glass substrate of controlled pore size. Nonspecific binding typically rarely occurs during hybridization on the silicon substrate, the quartz substrate, or the glass substrates. And, the glass substrates can transmit visible and/or UV light, so they are advantageous for detecting a fluorescent material. In addition, the silicon substrate and the glass substrates are advantageous in that various thin-film fabrication processes and photolithography that have been reliably established and used for the fabrication of semiconductor devices or liquid crystal display (LCD) panels can be used. Therefore, from the viewpoint of manufacturing process, the non-probe cell regions II may correspond to exposed silicon substrate surface or exposed glass substrate surface.

The linkers 140 have functional groups 142. The functional groups 142 of the linkers 140 have a probe-coupling reactivity higher than that of functional groups of the active pads 120 (e.g., SiOH), and may be made of a material capable of providing a space long enough for free interaction with biological samples. Formation of the linkers 140 may be skipped, when necessary.

The probes 160 may be probes according to the target of biological sample to be analyzed by the biochip 1. Useful examples of the probe include a DNA probe, a protein probe such as an antibody/antigen or a bacteriorhodopsin, a bacterial probe, a neuron probe, etc. According to the kind of probe used, the biochip may be a DNA chip, a protein chip, a cellular chip, a neuron chip, etc.

In exemplary embodiments of the disclosed technology, the probes 160 may be oligomer probes. As used herein, the term “oligomer” may mean a polymer formed of two or more monomers. In certain embodiments, the oligomer is covalently bound. In other embodiments, the polymer may have a molecular weight of up to about 1,000. In other embodiments, the oligomer may include from about 2 to about 500 monomers. In some embodiments, the oligomer may include from about 5 to about 30 monomers. However, the characteristics of the oligomer probe are not limited to the ranges listed above. The oligomer probes may be nucleosides, nucleotides, amino acids, peptides, etc.

As used herein, the terms “nucleosides” and “nucleotides” may include not only known purine and pyrimidine bases, but also methylated purines or pyrimidines, acylated purines or pyrimidines, etc. Furthermore, the “nucleosides” and “nucleotides” may include not only known (deoxy)ribose, but also a modified sugar which contains a substitution of a halogen atom or an aliphatic group for at least one hydroxyl group or is functionalized with ether, amine, or the like. As used herein, the term “amino acids” are intended to refer to not only naturally occurring, L-, D-, and nonchiral amino acids, but also modified amino acids, amino acid analogs, etc. As used herein, the term “peptides” refer to compounds produced by an amide bond between the carboxyl group of one amino acid and the amino group of another amino acid.

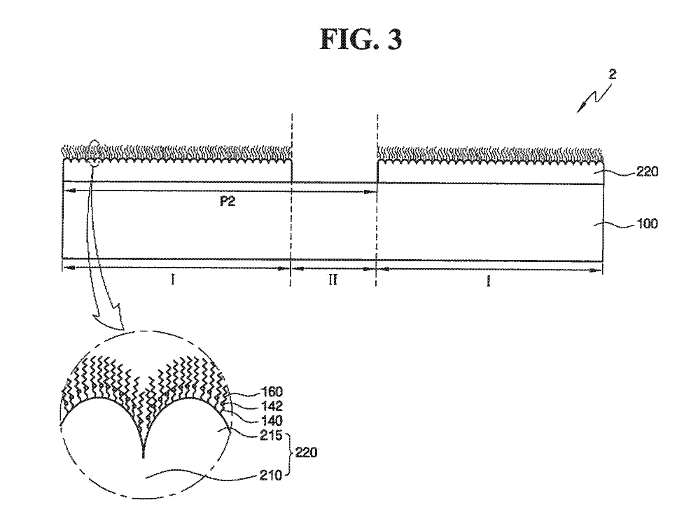

FIG. 3 is a sectional view of a biochip 2 according to a second embodiment of the disclosed technology.

Referring to FIG. 3, only one active pad 220 is formed for each probe cell region I, which can be applied when the biochip 2 is laid out such that a pitch P2 of the probe cell regions I can be n/4 (where n is an integer) times of a fluorescence wavelength (λ) for detection. In the biochip 2 according the second embodiment of the disclosed technology, the entire area of the probe cell regions I can be used as probe coupling areas. Materials of a base 210 and a surface unevenness 215 of the active pad 220 are substantially the same as those in the first embodiment, and a repeated explanation will not be given. In addition, the substrate 100, the linkers 140 and the probes 160 are substantially the same as those in the first embodiment, and a repeated explanation will not be given.

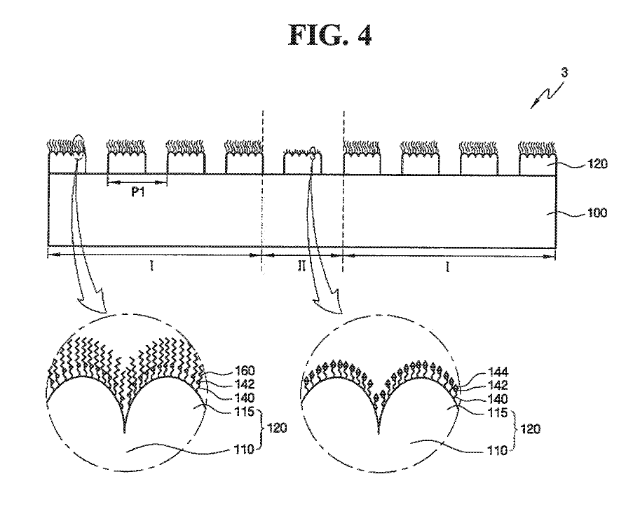

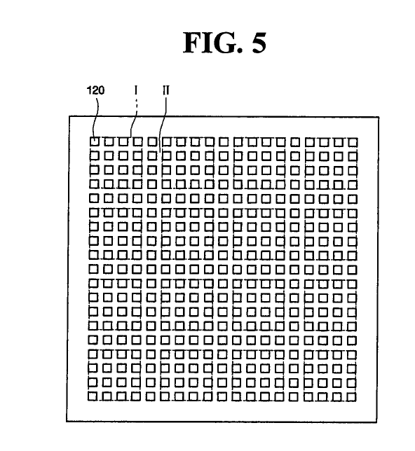

FIG. 4 is a sectional view of a biochip 3 according to a third embodiment of the disclosed technology, and FIG. 5 is a layout view of the biochip 3 shown in FIG. 4, according the third embodiment of the disclosed technology.

Referring to FIGS. 4 and 5, the biochip 3 according to a third embodiment of the disclosed technology is formed not only on probe cell regions I but also on non-probe cell regions II.

In a case where the non-probe cell regions II include active pads 120, linkers 140 are formed on the entire surface of the active pads 120. Probes 160 are selectively coupled only to the linkers 140 positioned in the probe cell regions I. Inactive capping groups 144 are coupled to ends of the linkers 140 positioned in the non-probe cell regions II. The biochip 3 is completed by activating only the probe cell regions I by photoactivation synthesis and coupling the probes 160 I thereto. In this case, the inactive capping groups 144 may be photolabile protecting groups. Since sizes, material, and patterns of the active pads 120 are substantially the same as those in the first embodiment, an explanation thereabout will not be given. In addition, the substrate 100, the linkers 140 and the probes 160 are substantially the same as those in the first embodiment, and a repeated explanation will not be given.

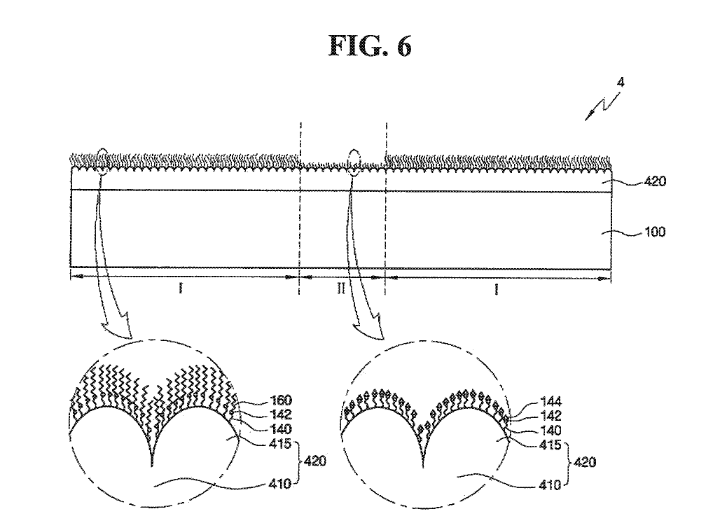

FIG. 6 is a sectional view of a biochip 4 according to a fourth embodiment of the disclosed technology.

Referring to FIG. 6, the biochip 4 according to a fourth embodiment of the disclosed technology includes an active layer 420 formed on the entire surface of a substrate 100, and probes 160 directly or indirectly coupled to the active layer 420.

The active layer 420 may be made of a material having substantially the same surface roughness as that of the active pads 120 of the first and second embodiments.

The active layer 420 is formed on the entire surface of the substrate 100 and includes a surface unevenness 415 to increase a surface area for coupling with the probes 160. The RMS (Root Mean Square) of the surface roughness 415 may range from about 0.2 nm to about 5 nm.

The active layer 420 may be made of materials that can provide functional groups by being directly or indirectly coupled to the probes 160. Alternatively, the active layer 420 may be made of materials that can provide the functional groups or by a variety of surface treatments, such as annealing, ozonolysis, acid treatment, or base treatment. The phrase “being directly coupled to the probes 160” means being coupled to the probes 160 without other intermediate substance, and the phrase “being indirectly coupled” means that being coupled to the probes 160 by means of the linkers 140.

The surface unevenness 415 forming the active layer 420 may be formed of silicon dots or hemispherical silicon grains (HSGs). When the surface unevenness 415 is formed of silicon dots, a base 410 of the active pads 120 may be made of silicon oxide. When the surface unevenness 415 is formed of hemispherical silicon grains (HSGs), the base 410 of the active pads 120 may be polysilicon or amorphous silicon.

The probes 160 are selectively coupled only to the linkers 140 positioned in the probe cell regions I. Inactive capping groups 144, e.g., photolabile protecting groups, are coupled to ends of the linkers 140 positioned in the non-probe cell regions II. The biochip 4 is completed by activating only the probe cell regions I by photoactivation synthesis and coupling the probes 160 thereto. Since the substrate 100, the linkers 140, and the probes 160 are substantially the same as those in the first embodiment, a repeated explanation will not be given.

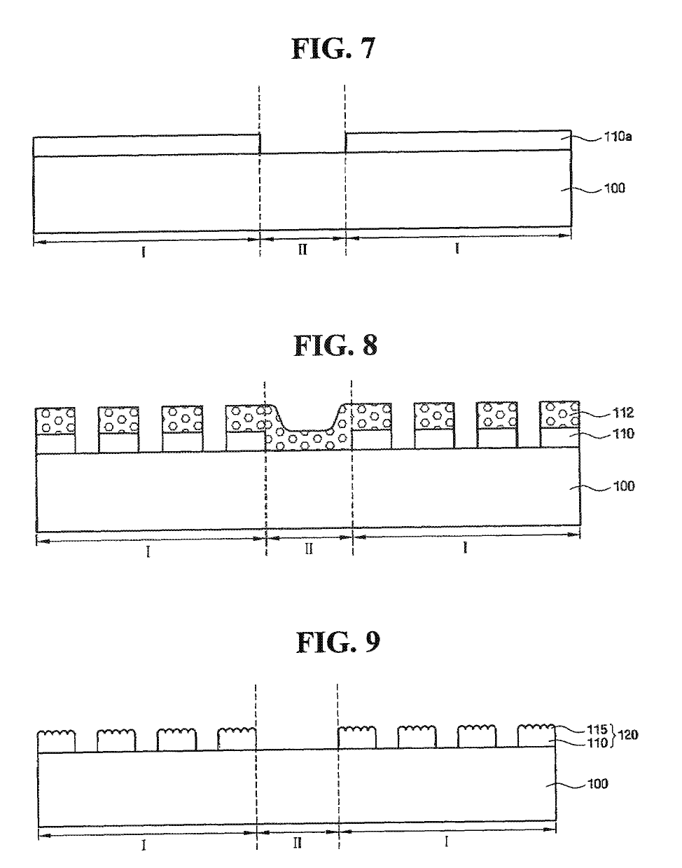

Hereinafter, a method of fabricating oligomer probe arrays according to exemplary embodiments of the disclosed technology will be described with reference to FIGS. 7 through 15.

FIGS. 7 through 11 are sectional views of intermediate structures illustrating a method of fabricating the biochip 1 illustrated in FIG. 1, according to the first embodiment of the disclosed technology.

Referring to FIG. 7, a silicon oxide film 110a is formed only on the probe cell regions I of a substrate 100. The silicon oxide film 110a may be a thermal oxide film formed by thermally oxidizing the substrate 100. A thickness of the silicon oxide film 110a is contributable to improvement of the resolution by forming the silicon oxide film 110a to become n-fourth (i.e., n/4) times of a fluorescence wavelength (λ) for detection, where n is an integer. The thickness of the silicon oxide film 110a is preferably in a range of about 100 nm to about 125 nm.

Referring to FIG. 8, an etch mask 112 is formed on the silicon oxide film 110a, and the silicon oxide film 110a is patterned to form a base 110 of active pads. Here, the patterning is performed such that a pitch P between the base 110 and it neighboring base becomes n/4 (n is an integer) times of a fluorescence wavelength (λ) for detection. For example, if the a fluorescence wavelength (λ) is in a range of about 400 nm to about 500 nm, the pitch between the bases may range from about 200 nm to about 250 nm.

Referring to FIG. 9, the etch mask 112 is removed, and the surface unevenness 115 formed of silicon dots is then formed on a top surface of the base 110. First the top surface of the base 110 is treated with a diluted HF solution. Subsequently, the substrate 100 is loaded into an RF PECVD (Radio Frequency Plasma Enhanced Chemical Vapor Deposition) chamber and supplies silane chloride (SiH₂Cl₂ or SiCl₄) as a source gas at low temperature in a range of, for example, 150 to 200° C., to form the surface unevenness 115 formed of silicon dots, thereby fabricating a biochip substrate having the active pads 120. When supplying the source gas, a small amount of hydrogen (H₂) gas may be supplied together with silane chloride (SiH₂Cl₂ or SiCl₄) gas, which allows crystallized silicon dots to be more effectively formed.

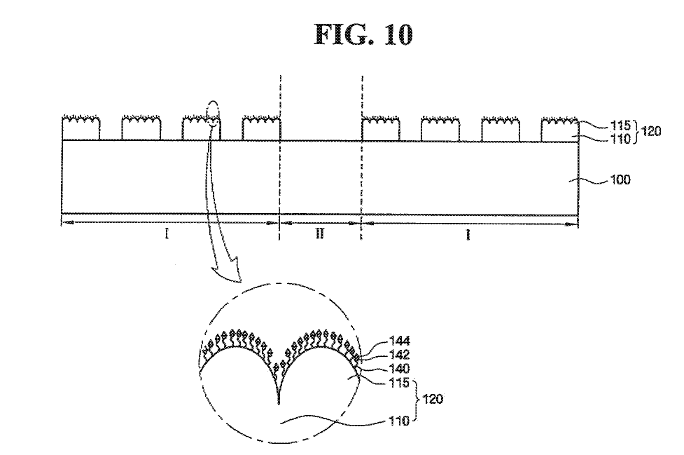

Referring to FIG. 10, optionally, in order to modify the surface of the active pads 120 so as to facilitate a reaction between the surface of the active pads 120 and the linkers 140, the active pads 120 are subjected to a surface treatment such as ozonolysis, acid treatment, base treatment, or combinations thereof. The annealing may be carried out in an oxygen atmosphere. The acid treatment may be carried out using, for example, a Piranha solution (a mixture of sulfuric acid and hydrogen peroxide). The base treatment may be carried out using an ammonium hydroxide solution.

Subsequently, the linkers 140 are formed on the surface treated active pads 120. Here, in a case where the probes 160 are synthesized using a photolithography process, which will be described below, photolabile groups 144 are attached to functional groups 142 of the linkers 140. The linkers 140 are selectively formed only on the active pads 120), while they are not formed on the exposed top surface of the substrate 100 in the non-probe cell region II.

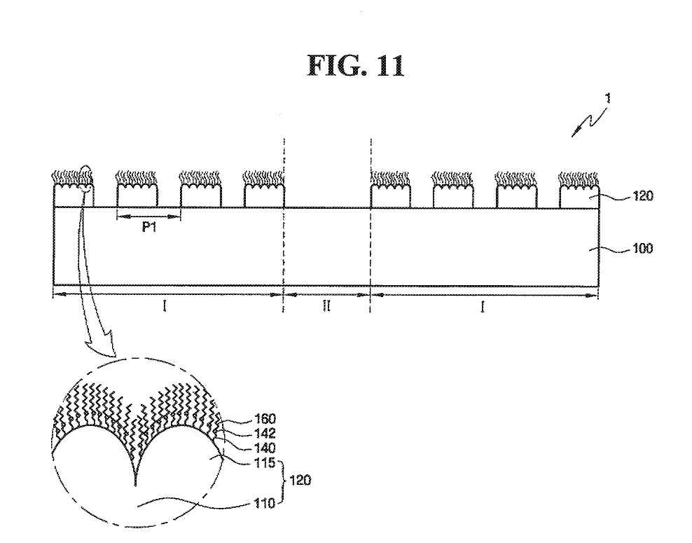

Referring to FIG. 11, the probes 160 are coupled to the linkers 140. When the functional groups 142 capable of being coupled with the probes at the ends of the linkers 140 are protected by the photolabile groups 144, selective exposure is performed for each probe cell region I to remove the photolabile groups 144 and the probes 160 are then coupled to the ends of the linkers 140. For example, the coupling of the probes 160 may be performed by spotting onto completed probes, or synthesizing monomers for probes (e.g., nucleotide phosphoamidite monomers having functional groups protected by photolabile groups) by photolithography. In this way, the biochip 1 according to the first embodiment of the disclosed technology is completed by forming the probes 160.

Unlike the method illustrated in FIGS. 7 through 9, the surface unevenness 115 may be formed by forming the silicon oxide film 110a on the entire surface of the substrate 100, and performing patterning such that the base 110 is formed only on the probe cell region I. Alternatively, the silicon oxide film 110a may be formed on the entire surface of the substrate 100 and the surface unevenness 115 is then formed, followed by patterning such that the active pads 120 are formed only on the probe cell region I.

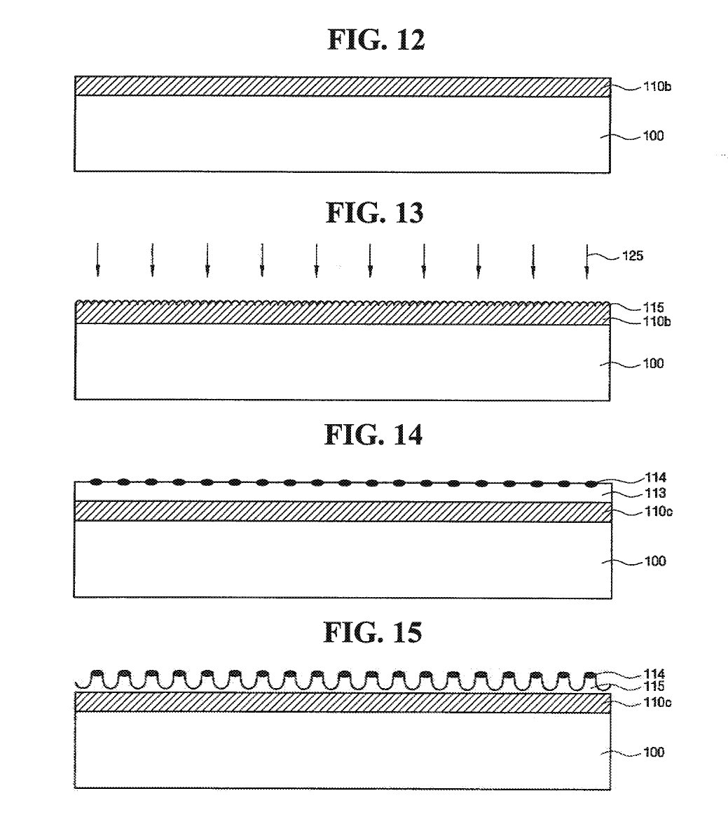

FIGS. 12 and 13 are sectional views of intermediate structures illustrating another method of fabricating the biochip illustrated in FIG. 1, according to the first embodiment of the disclosed technology.

Referring to FIG. 12, an amorphous silicon film 110b is first formed on the entire surface of the substrate 100.

Referring to FIG. 13, a two-step annealing process 125 is carried out to form unevenness 115 formed of hemispherical silicon grains (HSGs) on the amorphous silicon film 110b. In the first-step annealing is carried out by supplying silane (SiH₄) gas at a temperature in a range of about 550° C. to about 600° C. for about 10 to about 40 minutes, the second-step annealing is carried out in vacuum without source gas or inert gas supplied at a temperature in a range of about 500° C. to about 600° C. for about 1 to about 10 minutes, but the processing conditions are not limited to the illustrated conditions.

Thereafter, although not shown in the drawings, the amorphous silicon film 110b having the unevenness 115 is patterned, thereby forming the active pads 120 consisting of the base 110 and the unevenness 115 only on the probe cell regions I. Then, substantially the same processes as those shown in FIGS. 10 and 11 are performed to complete the biochip 1.

In some cases, the patterning of the amorphous silicon film 110b may be performed prior to the forming of the unevenness 115.

FIGS. 14 and 15 are sectional views of intermediate structures illustrating still another method of fabricating the biochip illustrated in FIG. 1, according to the first embodiment of the disclosed technology.

Referring to FIG. 14, an SOI (Silicon On Insulator) substrate consisting of a silicon substrate, a silicon oxide film 110c and a silicon film 113 sequentially stacked, is prepared, nano-sized silicon nitride nanomask 114 is formed on the silicon film 113 using a NANO LOCOS process. The silicon nitride nanomask 114 may be formed using an ultrahigh vacuum chamber maintained at about 750° C. in a nitrogen atmosphere.

Referring to FIG. 15, an etching mode oxidation process is performed using the silicon nitride nanomask 114 as an oxidation mask. The etching mode oxidation process may be carried out using an ultrahigh vacuum chamber maintained at about 870 psi in a low-pressure, oxygen atmosphere. During the etching mode oxidation process, the silicon film 113 exposed to the silicon nitride nanomask 114 is continuously etched to produce volatile SiO₂ molecules. As a result, the unevenness 115 formed of physically discrete silicon dots is produced.

Thereafter, although not shown in the drawings, the silicon oxide film 110c having the unevenness 115 is patterned, thereby forming the active pads 120 including the base 110 and having the unevenness 115 only on the probe cell regions I. Then, substantially the same processes as those shown in FIGS. 10 and 11 are performed to complete the biochip 1.

In some cases, the patterning of the silicon oxide film 110c may be performed prior to the forming of the unevenness 115.

The biochips 2, 3 and 4 shown in FIGS. 3, 4 and 6 can be fabricated by modifying some of the above-described steps shown in FIGS. 7 through 15. For example, the biochip 2 or 3 shown in FIG. 3 or 4 can be fabricated by modifying the patterning process such that only one active pad is formed only on each probe cell, or the active pads are formed on the entire surface of the substrate. Alternatively, the biochip 4 shown in FIG. 6 can be fabricated by forming the surface unevenness with the patterning process skipped. Accordingly, more concrete examples of the alternative embodiments can be deduced from the description made with reference to FIGS. 3, 4 and 6, and a detailed explanation will not be given.

The disclosed technology will be described in detail through the following experimental examples. However, the experimental examples are for illustrative purposes and other examples and applications can be readily envisioned by a person of ordinary skill in the art. Since a person skilled in the art can sufficiently analogize the technical contents which are not described in the following experimental examples, the description thereabout is omitted.

Unless otherwise specified in the following exemplary embodiments, the term “probe” is a DNA probe, which is an oligomer probe consisting of about 5-30 covalently bound monomers. However, the disclosed technology is not limited to the probes listed above and a variety of probes may used.

### EXPERIMENTAL EXAMPLE 1

### Formation of Active Pads on Probe Cell Region

A silicon nitride film was deposited on a silicon substrate to a thickness of 200 Å. A photoresist film was formed on the silicon nitride film to a thickness of about 1.2 μm using a spin-coating process and exposed to light for development with a pitch of 11 μm to form a photoresist pattern. The silicon nitride film was etched using the photoresist pattern as an etch mask to form a silicon nitride film pattern. The substrate was annealed at 900° C. for 1 hour using the silicon nitride film pattern as a mask to form a thermal oxide film having a thickness in a range of 0.9 to 100 nm at a portion of the silicon substrate exposed by the nitride film pattern.

Subsequently, a photoresist pattern, which exposes the thermal oxide film with a pitch of 200 nm while masking a non-probe cell region, is formed again. The thermal oxide film was patterned using the photoresist pattern as an etch mask to form thermal oxide film patterns arranged at a pitch of 200 nm on a probe cell region.

After removing the photoresist pattern, a surface of the thermal oxide film was treated with a hydrogen fluoride (HF) solution diluted with deionized water in 200:1, the substrate was loaded into an RF PECVD chamber, by supplying a source gas (e.g., silane chloride SiH₂Cl₂) for 30 seconds with RF power at 30 W, to form silicon dots on the top surface of the thermal oxide film pattern, thereby fabricating active pads arranged at a pitch of 200 nm on the probe cell region.

### EXPERIMENTAL EXAMPLE 2

### Formation of Active Pads on Entire Surface of Substrate

A silicon nitride film was deposited on a silicon substrate to a thickness of 200 Å. A photoresist film was formed on the silicon nitride film to a thickness of about 200 nm using a spin-coating process and exposed to light for development with a pitch of 200 nm to form a photoresist pattern. The silicon nitride film was etched using the photoresist pattern as an etch mask to form a silicon nitride film pattern. The substrate was annealed at 900° C. for 1 hour using the silicon nitride film pattern as a mask to form a thermal oxide film having a thickness in a range of 0.9 to 100 nm at a portion of the silicon substrate exposed by the nitride film pattern.

Subsequently, after a surface of the thermal oxide film was treated with a hydrogen fluoride (HF) solution diluted with deionized water in 200:1, the substrate was loaded into an RF PECVD chamber, by supplying a source gas (e.g., silane chloride SiH₂Cl₂) for 30 seconds with RF power at 30 W, to form silicon dots on the top surface of the thermal oxide film pattern, thereby fabricating active pads arranged at a pitch of 200 nm on the probe cell region.

### EXPERIMENTAL EXAMPLE 3

### Coupling of Probes

The substrates prepared in Experimental Examples 1 and 2 were annealed at 900° C. in an oxygen atmosphere for 3 hours to form a silanol (SiOH) group on surfaces of silicon dots. In order to facilitate binding between the silanol group and silane linkers, surfaces of the substrates were cleaned using a piranha solution (7:3 concentrated H₂SO₄/H₂O₂).

Subsequently, active pads were spin-coated with bis(hydroxyethyl)aminopropyltriethoxysilane at 500 rpm for 30 seconds, and stabilized at room temperature for about 5 to 30 minutes. Then, the resultant product was treated with an acetonitrile solution containing NNPOC-tetraethyleneglycol and tetrazole (1:1) so that phosphoramidite protected with photolabile groups was coupled, and then acetyl-capped, which resulted in completion of protected linker structures.

Next, ends of the linker structures are deprotected using a binary chrome mask and a 365 nm-wavelength projection exposure machine. The binary chrome mask exposes desired probe cell regions to light. And, it is applied an energy of 1000 mJ/cm² for one minute in the exposure machine. Then, the probe cell regions were treated with an acetonitrile solution containing amidite-activated nucleotide and tetrazole (1:1) to achieve coupling of the protected nucleotide monomers to the deprotected linker structures, and then treated with a THF solution (acetic anhydride (Ac20)/pyridine (py)/methylimidazole=1:1:1) and a 0.02M iodine-THF solution to perform capping and oxidation.

The above-described deprotection, coupling, capping, and oxidation processes were repeated to synthesize oligonucleotide probes having different sequences for each probe cell region.

As described above, in biochips and biochip substrates according to some embodiments of the disclosed technology, a reactive surface area for coupling of probes can be increased, and thus, the number of probes capable of coupling with each probe cell can be increased, compared to conventional biochips having the same design rule. Therefore, even when a reduced design rule is employed, desired detection intensity can be ensured.

In addition, in biochips and biochip substrates according to some embodiments of the disclosed technology, the active pads are arranged at a space allowing optical amplification, thereby selectively amplifying the wavelength of light used in data analysis of biochips and increasing the detection intensity.

While the present invention has been particularly shown and described with reference to exemplary embodiments thereof, it will be understood by those of ordinary skill in the art that various changes in form and details may be made therein without departing from the spirit and scope of the present invention as defined by the following claims. It is therefore desired that the present embodiments be considered in all respects as illustrative and not restrictive, reference being made to the appended claims to indicate the scope of the invention.

What is claimed is:

1. A biochip comprising:
a substrate;
a plurality of active pads formed on the substrate, each of the plurality of active pads having a surface roughness and being patterned so as to produce photonic crystals; and
a plurality of probes directly or indirectly coupled with at least some of the plurality of active pads.

2. The biochip of claim 1, wherein the surface roughness of each of the plurality of active pads has a Root Mean Square (RMS) within a range of about 0.2 nm to about 5 nm.

3. The biochip of claim 1, wherein the plurality of active pads are arranged at a pitch of n-fourth (n/4) times of a fluorescence wavelength (λ) for detection of the biochip, where n is an integer.

4. The biochip of claim 1, wherein the surface unevenness is formed of at least one of silicon dots and hemispherical silicon grains (HSGs).

5. The biochip of claim 1, wherein the biochip includes a plurality of probe cell regions to which probes are coupled, and non-probe cell regions to which probes are not coupled, the plurality of active pads are formed both on the plurality of probe cell regions and the non-probe cell regions, and the active pads formed on the non-probe cell regions are inactively capped without probes coupled thereto.

6. The biochip of claim 1, wherein the biochip includes a plurality of probe cell regions with which probes are coupled and non-probe cell regions with which probes are not coupled, and the plurality of active pads are formed only on the plurality of probe cell regions.

7. A biochip comprising:
a substrate;
a plurality of active pads or an active layer formed on the substrate, including a surface unevenness having a surface roughness of a Root Mean Square (RMS) within a range of about 0.2 nm to about 5 nm; and
a plurality of probes directly or indirectly coupled with at least a portion of the plurality of active pads.

8. The biochip of claim 7, wherein the surface unevenness comprises at least one of silicon dots and hemispherical silicon grains (HSGs).

9. The biochip of claim 7, wherein the plurality of active pads are arranged at a pitch of n-fourth (n/4) times of a fluorescence wavelength (λ) for detection of the biochip, where n is an integer.

10. The biochip of claim 7, wherein the biochip includes:
a plurality of probe cell regions, wherein at least a portion of the plurality of probes are coupled with at least a portion of the plurality of probe cell regions; and
a plurality of non-probe cell regions, wherein the plurality of probes are not coupled with the plurality of non-probe cell regions,
wherein the plurality of active pads are formed both on the plurality of probe cell regions and the plurality of non-probe cell regions, and wherein the active pads formed on the non-probe cell regions are capped and are not coupled to the plurality of probes.

11. The biochip of claim 7, further comprising:
a plurality of probe cell regions coupled with the plurality of probes; and
a plurality of non-probe cell regions which are not coupled with the plurality of probes,
wherein the active pads are formed on the plurality of probe cell regions.

12. A biochip substrate comprising a plurality of active pads having a surface unevenness, wherein the plurality of active pads are patterned so as to produce photonic crystals, and wherein the plurality of active pads are capable of being directly or indirectly coupled to functional groups that are coupled to probes.

13. The biochip substrate of claim 12, wherein the plurality of active pads include a surface roughness having a Root Mean Square (RMS) of about 0.2 nm to about 5 nm.

14. The biochip substrate of claim 12, wherein the plurality of active pads are arranged at a pitch of n-fourth (n/4) times of a fluorescence wavelength (λ) for detection of the biochip substrate, where n is an integer.

15. The biochip substrate of claim 12, wherein the surface unevenness is formed of at least one of silicon dots and hemispherical silicon grains (HSGs).

16. A biochip substrate comprising a plurality of active pads or an active layer including a surface unevenness including a surface roughness having a Root Mean Square (RMS) of about 0.2 nm to about 5 nm, wherein the plurality of active pads is capable of producing functional groups directly or indirectly coupled to probes attached to a surface of the biochip substrate.

17. The biochip substrate of claim 16, wherein the surface unevenness is formed of at least one of silicon dots and hemispherical silicon grains (HSGs).

18. The biochip substrate of claim 16, wherein the plurality of active pads are arranged at a pitch of n-fourth (n/4) times of a fluorescence wavelength (λ) for detection of the biochip substrate, where n is an integer.
